(19) United States
(12) Patent Application Publication
(10) Pub. No.: US 2023/0287319 A1
(43) Pub. Date: Sep. 14, 2023

(54) PETRI DISH

(71) Applicant: ReproHealth Technologies, Inc., Indianapolis, IN (US)

(72) Inventors: James Donahue, Indianapolis, IN (US); Jason Swain, Lone Tree, CO (US); Michael D. Whitt, San Luis Oblspo, CA (US); Katherine Russeil, Lebanon, IN (US); Nishanth Reddy Narayan, Pleasanton, CA (US); Catherine Amber Cole, Cupertino, CA (US); Jasmine Janisch, San Luis Obispo, CA (US)

(21) Appl. No.: 18/120,674

(22) Filed: Mar. 13, 2023

Related U.S. Application Data
(60) Provisional application No. 63/319,371, filed on Mar. 13, 2022.

(51) Int. Cl. C12M 1/32; C12M 1/00

(52) U.S. Cl. CPC C12M 23/12; C12M 23/38; C12M 23/34

(57) ABSTRACT

An apparatus includes a cellular culture container having an interior cavity. A cellular culture area is located in the interior cavity. The cellular culture area is configured to receive at least one cell to be cultured. A condensation director is configured to reduce and/or prevent the entry of condensation into the cellular culture area.

### CROSS-REFERENCE TO RELATED APPLICATIONS

The present application claims the benefit of U.S. Provisional Application No. 63/319,371, filed Mar. 13, 2022, the entirety of which is herein incorporated by reference.

### TECHNICAL FIELD

The present application generally relates to cellular culture, and more specifically, but not exclusively, to containers for cellular culture.

### BACKGROUND

In the late 1800s, Julius Richard Petri invented a glass dish in which bacterial cells could be cultured. Today, a variety of cells are cultured in single-use plastic petri dishes. Petri dishes are utilized to contain various cells to be cultured, such as yeast, bacteria, fungi, etc. and a suitable culture medium can be contained within the petri dish.

FIG. 1 depicts an exemplary petri dish 100 of the prior art. This petri dish 100 is a 60 mm dish manufactured by Falcon. The petri dish 100 includes a base 102 having a lower surface 108 and a sidewall 106. A lid 104 is configured to removably couple to the base, thereby forming an enclosure.

Although petri dishes of the prior art function in a workmanlike manner, they include numerous drawbacks. For example, in certain conditions condensation can form on the interior of the petri dish, especially on the interior surface of the lid. This condensation can freely drip from the lid into the culture, which may contaminate the culture and/or obscure cell visualization.

Assisted reproductive technologies, including in vitro fertilization (hereinafter “IVF”), are commonly utilized by those suffering infertility. In traditional IVF, embryos are cultured in flat bottomed petri dishes. The gametes and/or embryos housed in these petri dishes are placed in embryonic culture media, which is then often overlaid with mineral oil.

Once the dish is prepared and the cells loaded, the petri dishes are placed in laboratory incubators which maintain the temperature and pH of the embryonic culture medium and the embryos housed therein. The incubators are commonly set at 37° C. and are filled with a premixed gas comprising approximately 89% Nitrogen, 5% Oxygen, and 6% Carbon Dioxide. This specific temperature and gas concentration enables an adequate pH of the culture media to be maintained and is designed to simulate the in vivo conditions thought to be required for early embryonic development.

To reduce the likelihood of contamination by bacteria, dry culture (e.g., in which the incubator does not include a humidification system) is commonly utilized. In such dry culture systems, culture medium can evaporate, even with an oil overlay. This evaporation results in an increased osmolality within the culture medium, which can be detrimental for embryonic development.

Therefore, further technological developments are desirable.

### SUMMARY

One form of the present application is directed to an apparatus that includes a cellular culture container having an interior cavity. A cellular culture area is located in the interior cavity. The cellular culture area is configured to receive at least one cell to be cultured. The apparatus includes a condensation director.

The apparatus can include a reservoir that is physically separated from the cellular culture area. The condensation director can be configured to direct the condensation toward the reservoir.

The cellular culture container can include a lid configured to removably cooperate with the base. The condensation director can be located at the lid. The condensation director can be integrally formed with the lid. A lower surface of the lid can at least partially define the condensation director.

A reservoir can be located in the base. The condensation director can at least partially extend over the cellular culture area and the condensation director can include an inclined surface. The condensation director can be configured to provide for the flow of condensation along the inclined surface toward the reservoir.

The inclined surface can be disposed an angle that is greater than 3 degrees. The inclined surface can include a downwardly directed curvature.

A sealing member can be located between the base and the lid. The reservoir can include an evaporative humidifier. The sealing member can be configured to prevent the egress of water vapor from the interior cavity.

Another form of the present application is directed to a petri dish which includes an outer housing having an interior cavity. A cell culture stage is located in the interior cavity. The cell culture stage is configured to receive a culture. A condensation director is configured to prevent condensation from entering the culture.

The petri dish can include an evaporative humidifier configured to humidify a substantially sealed atmosphere located in the interior cavity. The outer housing can include a base and a lid. A reservoir can be located at the base. The reservoir can be physically separated from the cell culture stage.

The condensation director can be integrally formed with the lid. The condensation director can at least partially extend over the cell culture stage. The condensation director can include an inclined surface. The condensation director can be configured to direct condensation into the reservoir. The condensation director is configured to prevent condensation from dripping into the cell culture stage.

The inclined surface can include a curvature. The cellular culture stage and reservoir can be integrally formed in the base. The reservoir can be located at a central portion of the base.

The petri dish can include a reservoir wall defining an outer perimeter of the reservoir. The cellular culture stage can include a plurality of culture wells. Each culture well can be configured to receive a suitable culture medium and at least one of a gamete, a fertilized gamete, and/or an embryo. The culture wells can be separated by a plurality of dividers which extend between the reservoir wall and an outer wall of the base.

A height dimension of the dividers can be less than a height dimension of the reservoir wall.

Yet another form of the present application is directed to a petri dish which includes a lid configured to sealingly couple with a base. An internal cavity is at least partially defined between the lid and the base. The lid and the base sealingly cooperate to reduce and/or prevent the egress of water vapor from the internal cavity to an external environment. A cell culture stage is located in the internal cavity. The cell culture stage is configured to receive at least one of a gamete, a fertilized gamete, and/or an embryo. A humidifier is in flow communication with the internal cavity.

The humidifier can be a reservoir. The reservoir can be located in a central portion of the base.

The petri dish can include a condensation director located at a lower surface of the lid. The condensation director can include an inclined surface which extends downwardly toward the central portion of the base. The condensation director is configured to direct condensation into the reservoir.

The condensation director can at least partially extend over the cell culture stage. The condensation director can provide for condensation to flow downwardly along the inclined surface absent condensation dripping from the inclined surface onto the cell culture stage.

The cell culture stage can be integrally formed in the base. The condensation director can be integrally formed in the lid. A sealing member can be disposed between the base and the lid.

Yet a further form of the present application is directed to a container for culturing cells. The container includes a base having an interior cavity. A cell culture stage is located internal to the interior cavity. The cell culture stage is configured to receive at least one cell to be cultured. A lid is configured to be removably received on the base. A condensation director is located at an inner surface of the lid. The condensation director is configured to direct condensation forming on the inner surface of the lid to a location in the interior cavity which is physically separated from the culture medium.

The condensation director can be integrally formed with the lid. The condensation director can include a downwardly directed angle on the inner surface of the lid, and the condensation director can extend downwardly toward a center of the lid.

The cell culture stage can be configured to be removably received within the interior cavity of the base. The cell culture stage can include a plurality of wells. Each well can be configured to receive culture medium and at least one cell to be cultured.

A reservoir can be centrally located in the base. The plurality of wells can be located in a channel that at least partially surrounds the reservoir. The channel can be at least partially defined by the cell culture stage and the condensation director can configured to direct condensation into the reservoir.

The reservoir can be configured to maintain and/or increase the humidity of an atmosphere confined within the internal cavity.

Each well can be configured to receive at least one of a gamete, a fertilized gamete, and/or an embryo, as well as embryonic culture medium. An oil overlay can be located atop the embryonic culture medium.

Other forms of the present application include unique cell culture apparatuses, devices, systems, and methods. Further embodiments, inventions, forms, objects, features, advantages, aspects, and benefits of the present application are otherwise set forth or become apparent from the description and drawings included herein.

### BRIEF DESCRIPTION OF THE DRAWINGS

The description herein makes reference to the accompanying drawings wherein like reference numerals refer to like parts throughout the several views, and wherein:

FIG. 1 depicts a petri dish of the prior art;

FIG. 2 is a cross-sectional view of an exemplary humidified petri dish according to a first form of the present application;

FIG. 3 is a perspective view of the petri dish of FIG. 1, depicted as having a transparent construction;

FIG. 4 is an exploded view of the petri dish;

FIG. 5 is a cross-sectional view of an exemplary petri dish lid, depicting an exemplary condensation director integrally formed with the lid;

FIG. 6 is a cut-away perspective view of an exemplary, removable cell culture stage;

FIG. 7 is a top view of the cell culture stage, depicted inserted into a base of the petri dish;

FIG. 8 is a perspective view of FIG. 7;

FIG. 9 is a side view of an exemplary humidified petri dish, according to another form of the present application, the petri dish depicted as including a transparent construction;

FIG. 10 is a cross-sectional view of an exemplary petri dish according to yet a further form of the present application; and

FIG. 11 is a perspective view of an exemplary petri dish base, the exemplary base having a reservoir and cell culture stage formed therein.

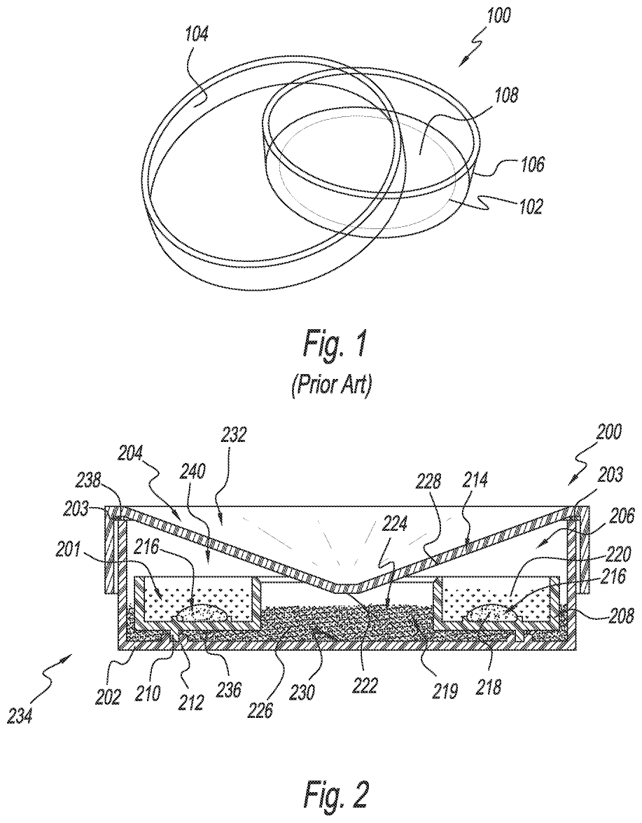

The accompanying drawings incorporated in and forming a part of the specification illustrate various forms and features of the present application; however, the present application should not be construed as being limited to those specific embodiments depicted in the drawings.

### DETAILED DESCRIPTION

For purposes of promoting an understanding of the principles of the invention, reference will now be made to the embodiments illustrated in the drawings and specific language will be used to describe the same. It will nevertheless be understood that no limitation of the scope of the invention is thereby intended, any alterations and further modifications in the illustrated device, and any further applications of the principles of the invention as illustrated therein being contemplated as would normally occur to one skilled in the art to which the invention relates.

Referring now to FIG. 2, an exemplary petri dish 200 according to a first form of the present application will now be described. As utilized herein, the term “petri dish” includes any dish, housing, enclosure, container, and/or vessel suitable for cellular culture. The petri dish 200 includes a base 202 and a lid 204. An interior cavity 206 is defined between a lower surface 228 of the lid 204 and an interior surface 230 of the base 202. The interior cavity 206 is an enclosed space when the lid 204 is placed upon the base 202.

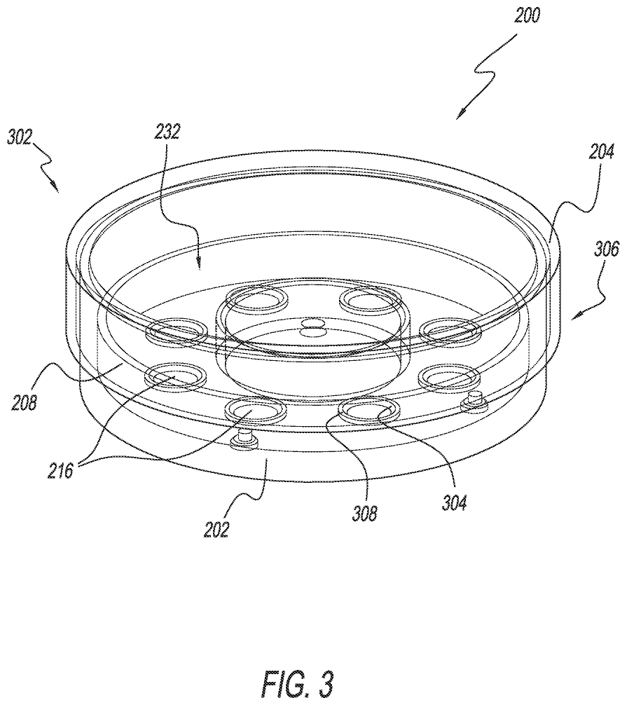

Referring to FIGS. 2-4, the base 202 includes an interior surface 230 and a sidewall 404 which extends upwardly from the interior surface 230. As is illustrated, the base 202 can include a substantially dish-type shape 424. A cellular culture area 201 is located in the interior cavity 206 of the petri dish 200. The cellular culture area 201 is depicted as being positioned within the base 202. The cellular culture area 201 receives the cells to be cultured within the petri dish 200.

Referring now to FIGS. 2-5, an exemplary lid 204 will now be described. An outer perimeter of the lid 204 is depicted as including a downwardly extending sidewall 402. An enclosed internal cavity 206 is formed in the petri dish 200 when the lid 204 is placed on the base 202. Placement of the lid 204 on the base 202 serves to contain humidity (e.g., water vapor within the atmosphere 240) within the enclosed internal cavity 206. In this closed configuration 302, the downwardly extending sidewall 402 of the lid 204 is located outwardly from the upwardly extending sidewall 404 of the base 202. Toward the outer perimeter of the lid 204, a lower surface 228 of the lid 204 can rest upon an upper rim 238 of the sidewall 404 of the base 202.

A sealing member 203 can provide sealing engagement between the lid 204 and the base 202. Cooperation between the lid 204, the base 202, and the sealing member 203 will substantially vapor seal the internal cavity 206 (e.g., water vapor within the atmosphere 240 is confined within the internal cavity 206). Reducing and/or eliminating the egress of water vapor from the internal cavity 206 can reduce decreases in the humidity of the atmosphere 240 (e.g., significant reductions in humidity are known to occur within petri dishes disposed in dry incubators). Moreover, vapor sealing the internal cavity 206 is believed to significantly increase the efficacy of any humidifier which is integrated into the petri dish 200, as will be discussed hereinafter.

One or more sealing members 203 can be located on the upper rim 238 of the sidewall 404 of the base 202 and/or can be located on the lower surface 228 of the lid 204 to provide sealing engagement between the lid 204 and the base 202. When the lid 204 is placed on the base 202, the sealing member 203 is sealingly engaged between the upper rim 238 of the base 202 and the lower surface 228 of the lid 204. Additionally and/or alternatively, a sealing member 203 can be located between the sidewall 404 of the base 202 and the downwardly extending sidewall 402 of the lid 204. The sealing member 203 can take the form of a silicone ring or silicone sealing tape; however, the use of various seals and sealing members is contemplated herein.

Although the internal cavity 206 is preferably vapor sealed, the lid 204 and base 202 can include a gas permeable construction (e.g., constructing the base 1006 and/or the lid 1004 from polystyrene or other polymers can yield a petri dish 200 which is carbon dioxide (hereinafter “CO2”) permeable, which can help regulate the pH of culture medium within the cell culture area 201 in IVF applications). The sealing member 203 can include a Teflon construction, such as Teflon tape. It is been discovered that Teflon tape has high CO2 permeability, but prevents the passage of water vapor therethrough. In this gas permeable form, although water vapor is confined within the internal cavity 206, CO2 can flow into or out from the internal cavity 206 to an atmosphere external to the petri dish 200.

The lid 204 can include a condensation director 232. This condensation director 232 is depicted as located at the lower surface 228 of the lid 204. The condensation director 232 includes an inclined surface 214, which is placed at an angle 514. The condensation director 232 serves as a flowpath for condensation 508 forming on the lower surface 228 of the lid 204 to pass over the culture in the cellular culture area 201 without dripping therein. In this manner, any condensation droplets 508 which form on the lower surface 228 of the lid 204 will not drip into the cellular culture area 201, preventing contamination of the culture housed therein. The condensation director 232 can direct condensation droplets into a reservoir 224 which is physically separated from the cellular culture are 201.

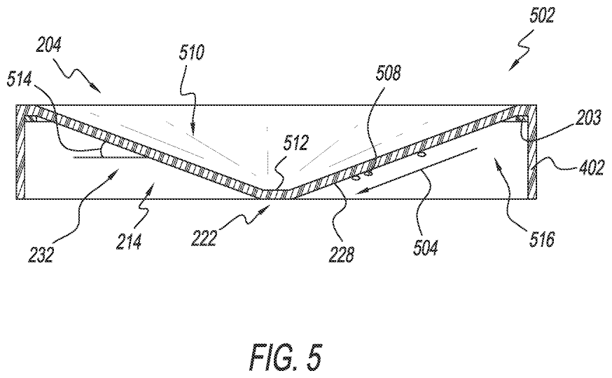

As is illustrated in FIG. 5, the condensation director 232 is formed through the integration of an inclined surface 214 at the lower surface 228 of the lid 204. This inclined surface 214 is oriented at angle 514. The magnitude of the angle 514 should be sufficient such that condensation droplets 508 will flow along the lower surface 228 of the lid 204 (e.g., following the direction of flow 504 for the right-side portion 516 of the lid 204), with the condensation droplets 508 hanging from and remaining adhered to the lower surface 228 of the lid 204. Selecting an angle 514 of sufficient magnitude is believed to substantially reduce and/or prevent condensation 508 from dripping from the lower surface 228 of the lid 204 into the culture below. This reduction and/or elimination of condensation 508 drippage from the lower surface 228 is believed to occur as the angle 514 of the condensation director 232 causes the condensation droplets 508 to flow downwardly along the lower surface 228, due to the force of gravity acting on the droplets 508, prior to the condensation droplets 508 collecting as larger droplets (e.g., through cohesion) which will eventually drip from the lower surface 228 of the lid 204 into cellular culture area 201 below.

It is believed that an angle 514 which is greater than 3 degrees can be of sufficient magnitude; however, angles selected from the range of 2 degrees to 45 degrees may be of sufficient magnitude. Although angles in the range of 45 degrees to almost 90 degrees will enable the condensation director 232 to function correctly; however, such large angles can result in a significant increase in a height of the petri dish 200. The exemplary, non-limiting angle 514 depicted in FIG. 5 approximates 20 degrees. It is important to note that the aforementioned angles are merely exemplary, and verification testing has not yet been performed.

The inclined 214 lower surface 228 extends above and over the cellular culture area 201. Condensation 508 forming on the lower surface 228 of the lid 204 will flow along the lower surface 228 of the lid 204 parallel to flow direction 504, and will hang from the lower surface 228 of the lid 204 without dripping into the cellular culture area 201 below. The incorporation of condensation director 232 into the petri dish 200 is believed to substantially reduce and/or prevent condensation 508 from dripping into the cellular culture area 201 and contaminating the culture therein.

The condensation director 232 can include a substantially frustoconical cross-sectional profile (e.g., with the frustoconical shape extending from the outer perimeter of the lid 204 downwardly and inwardly toward a center 512 of the lid 204). Condensation 508 which forms on the lower surface 228 of the lid 204 will flow downwardly along the lower surface 228 toward the center 512 of the lid 204. The condensation 508 will collect at the central surface 222 of the frustoconical shape and will drip off the central surface 222 into the reservoir 224, located below. The central surface 222 is depicted as being the lowermost surface of the condensation director 232.

The upper surface 510 of the lid 204 can include a substantially funnel type shape 502, which is defined by an upper surface of the condensation director 232. Such a construction is believed to reduce the amount of material utilized in the construction of the lid 204 (e.g., relative including a substantially flat upper surface 510 above the condensation director 232.

However, use of an inverted condensation director (not shown) is contemplated herein. In such an inverted condensation director, the frustoconical shape is inverted (e.g., with the highest surface of the frustoconical shape being located toward a center of the lid, and in which the lower surface of the lid is angled downwardly toward an outer perimeter of the lid). In this inverted design, in which the central surface is the highest point of the condensation director, condensation will flow along the lower surface of the lid toward the outer perimeter of the lid. A reservoir can be located in the base toward an outer perimeter of the base, with a cellular culture area located toward a central portion of the base.

The condensation director can alternatively take the form of an angled surface extending completely across the lid (e.g., from a high point at the right side to a low point at the left side) with a reservoir located in the base below the low point at the left side.

The cellular culture area 201 can take the form of a cellular culture stage 208. The cells to be cultured within the interior cavity 206 can be placed on the cellular culture stage 208. The cellular culture stage 208 is physically separated from the reservoir 224. Although it is contemplated that the cellular culture stage 208 can be integrally formed into the base 202, as will be discussed hereinafter, the cellular culture stage 208 is depicted as being removably insertable into the base 202.

Referring now to FIGS. 2-4, the cellular culture stage 208 can include a substantially disc-like, donut-like shape 406. The cellular culture stage 208 is depicted as including an outer wall 410, an inner wall 412, and a lower interior surface 408 which extends between the outer wall 410 and the inner wall 412. An opening 416 is located radially inwardly from the inner wall 412. A channel 414 is defined between the outer wall 410 and the inner wall 412. The culture can be placed within the channel 414 on the lower interior surface 408.

The cellular culture stage 208 can include one or more wells 216. Although the cellular culture stage 208 is depicted as including eight wells 216, it is contemplated that any number of wells 216 may be included in the cellular culture stage 208. Each well 216 is depicted as being defined by an outer wall 308 which extends upwardly from the lower interior surface 408. The wells 216 can include a fillet 304 between the lower interior surface 408 and the outer wall 308.

The wells 216 are configured to house the culture as well as a suitable culture medium if desired. The culture is confined by the sidewall 308 of the well 216. When utilized in connection with IVF, an oil overlay 220 can be placed in the channel 414, with the oil resting above the embryonic culture medium 218 and gametes and/or embryos. IVF is discussed herein as an exemplary application for the petri dish 200; however, the present petri dish 200 can be utilized to culture a variety of cells, including, but not limited to bacteria, yeasts, molds, various human and animal cells (e.g., stem cells, embryos, etc.) and the like.

The cellular culture stage 208 can be removably inserted into the base 202. As is best shown in FIG. 2, a plurality of legs 210 can extend downwardly from a lower surface 236 of the cellular culture stage 208. The lower interior surface 230 of the base 202 can include a number of receiving members 212. When the cellular culture stage 208 is inserted into the base 202, a distal end of each leg 210 is inserted into and received by a receiving member 212. The cooperation between the legs 210 and receiving members 212 can maintain the position of the cellular culture stage 208 relative the base 202 during typical movement of the petri dish 200 in a laboratory environment. The legs 210 can elevate the lower surface 236 of the cellular culture stage 208 above the lower interior surface 230 of the base 202.

When the lid 204 is placed atop the base 202, the central surface 222 of the condensation director 232 can extend below an upper rim of the inner wall 412 of the culture stage 208. The central surface 222 is depicted as being the lowermost surface, or distal end, of the condensation director 232. Condensation 508 which collects on the lower surface 228 will flow downwardly along the lower surface 228 toward the central surface 222, over the channel 414 of the culture stage, and will drip from the central surface 222 through the opening 416 and into the reservoir 224.

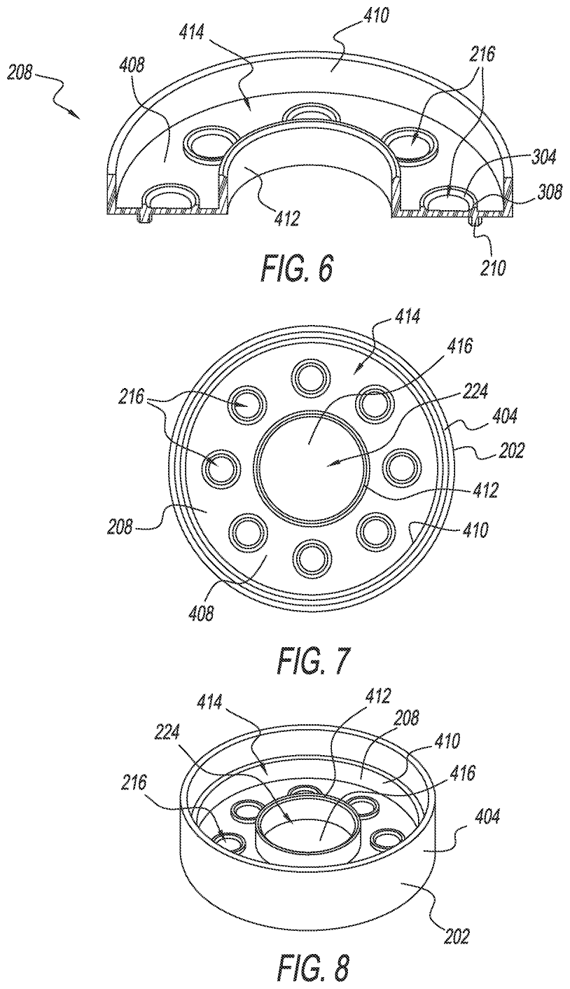

FIG. 6 depicts a partial cut-away perspective view of the cellular culture stage 208. FIG. 7 depicts a top view of the cellular culture stage 208 inserted into the base 202. FIG. 8 depicts a perspective view of the cellular culture stage 208 inserted into the base 202. As is illustrated, when the cellular culture stage 208 is inserted into the base 202, the outer wall 410 of the cellular culture stage 208 is located internal to the sidewall 404 of the base 202.

In further forms (e.g., as will be discussed hereinafter with regard to FIG. 11), it is contemplated that a cellular culture stage can be integrally formed with the base. In such an integral design, the inner wall of the cellular culture stage can extend upwardly from, and be interconnected with, a lower interior surface of the base. In this integral design, the inner wall can serve as a physical separation between a water reservoir and the culture. Wells can be formed into the lower interior surface of the base.

The petri dish 200 can include a humidifier 219 to humidify the atmosphere within the petri dish 200. As utilized herein, the term humidifier is intended to encompass a variety of devices which can increase the humidity (e.g., the moisture content and/or water vapor present) of an atmosphere, and the term humidifier includes powered humidification as well as unpowered evaporative humidification.

The humidifier 219 is depicted as taking the form of a water reservoir 224, which serves as an evaporative humidifier 219. As is best illustrated in FIG. 2, water 226 which is contained within the water reservoir 224 evaporates into the atmosphere 240 within the interior cavity 206. This evaporation of water 226 humidifies the atmosphere 240 (e.g., increasing the water vapor content of the atmosphere 240) within the interior cavity 206. It is believed that the inclusion of the humidifier 219 within the petri dish 200 can provide a more suitable environment for cellular culture. Additionally, it is believed that by maintaining and/or increasing the humidity within the interior cavity 206, the evaporation of water from the culture media 218 can be reduced, thereby maintaining the osmolality of the culture medium 218. It is believed that the inclusion of the humidifier 219 within the petri dish 200 can preserve the osmolality of the culture medium 218 within 5 percent over a typical IVF culture period (e.g., the osmolality of the culture medium 218 will not deviate more than 5 percent during the typical IVF culture period).

The reservoir 224 is depicted as being at least partially surrounded by the inner wall 412 of the cellular culture stage 208. The legs 210 of the cellular culture stage 208 can elevate the cellular culture stage 208 above the lower interior surface 230 of the base 202. In this form, the humidifier 219 is substantially a water bath with the cellular culture stage 208 disposed therein.

As is best illustrated in FIG. 2, water 226 within the reservoir 224 can surround the channel 414 of the cellular culture stage 208. Water 226 is depicted as being present below the lower surface 236 of the cellular culture stage 208, between the outer wall 410 of the cellular culture stage 208 and the sidewall 404 of the base 202, and in the opening 416 defined by the inner wall 412.

As will be appreciated, in the case of an evaporative humidifier 219, an overall surface area of the reservoir 224 which is in contact with the atmosphere 240 will impact the rate of evaporation of water 226 into the atmosphere 240. The reservoir 224 can include a sufficiently sized surface area to provide the desired level humidity to the atmosphere 240 within the interior cavity 206.

To fill the humidifier 219, the lid 204 is removed from the base 202 and water 226 can be poured through the opening 416 into the reservoir 224. The water 226 will rest upon the lower interior surface 230 of the base 202. A water 226 fill height within the base 202 should not exceed an upper height of the walls 410, 412 of the cellular culture stage 208. This fill height prevents water 226 from flowing and/or spilling from the reservoir 224, over the walls 410, 412, and into the channel 414 of the cellular culture stage 208.

It is contemplated that the humidifier can take the form of a super absorbent polymer which is placed in flow communication with the internal cavity of a petri dish. Water can be introduced into the super absorbent polymer, which will absorb and retain the water. The water present within the super absorbent polymer will evaporate into the atmosphere of the interior cavity of the petri dish, increasing the humidity within the interior cavity. It is contemplated that the super absorbent polymer can be placed at a number of locations within the interior cavity, such as within the base or internal to the lid.

Although the petri dish 200 has been described herein as having a substantially cylindrical, puck-like shape 306, it is also contemplated that the petri dish 200 (e.g., including the base 202, lid 204, and culture stage 208) can take a variety of forms, which include, but are not limited to, rectangular, square, or ovaloid. It is also contemplated that if the petri dish includes a rectangular form, the condensation director may include a substantially pyramidal shape. The petri dish 200 has been described as including a condensation director 232, a cellular culture stage 208, and a humidifier 219, however, it is contemplated that petri dishes 200 embodying the teachings of the present application may include one or more of these features, depending upon the desired design parameters of the desired petri dish.

The base 202 can be formed of a polymer 422. The cellular culture stage 208 can be formed of a polymer 420, and the lid 204 can be formed of a polymer 418. Preferably, the polymers 418, 420, and 422 are polystyrene, and the base 202, cellular culture stage 208, and lid 204 can be formed through injection molding. To enable ease of viewing the culture and water level, the petri dish 200 can be substantially transparent. The petri dish 200 can be prepackaged in sterile medical packaging, and can be single use (e.g., designed to be discarded after the desired culture is complete). However, it is also contemplated that the base 202, lid 204, and cellular culture stage 208 may be formed utilizing a variety of forming techniques, from a variety of polymers or glasses, as are known within the art of petri dish construction.

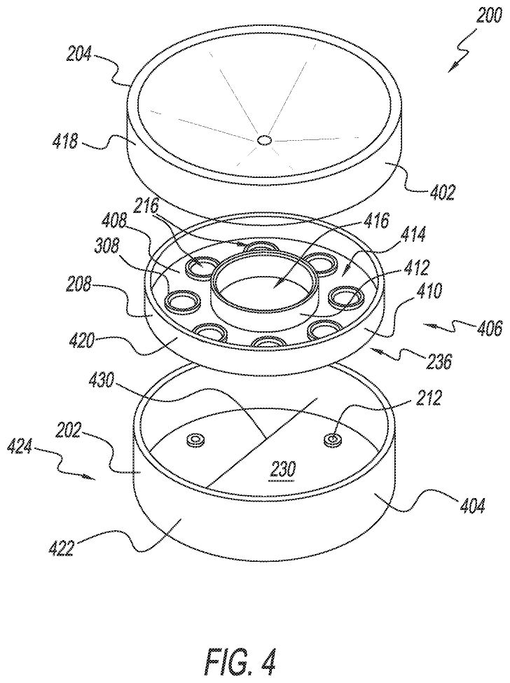

Referring back to FIGS. 2 and 4, one exemplary, non-limiting method for the loading and use of the petri dish 200 in connection with IVF will now be described. However, as has been previously described herein, a variety of cells may be cultured within the petri dish 200. It should be appreciated that desirable conditions for cellular culture to occur, which can include, but are not limited to temperature, atmospheric composition, etc. will depend upon the specific cells to be cultured.

The petri dish 200 can be removed from a sterilized package by a practitioner. The base 202 can be placed on a flat work surface, with the sidewall 404 extending upwardly. The culture stage 208 is then placed in the base 202. The legs 210, which extend downwardly from the lower surface 236 of the culture stage 208, are aligned with and inserted into the receiving members 212, located at the lower surface 230 of the base 202.

The practitioner can then place a suitable embryonic culture medium 218 into the wells 216 of the cell culture stage 208. As can be appreciated, the number of wells 216 to be at least partially filled with embryonic culture medium 218 and gametes and/or embryos can depend upon the desired number of gametes and/or embryos to be cultured within the petri dish 200.

The practitioner can place a suitable oil overlay 220 into the channel 414 of the culture stage 208. As is known, the oil 220 overlay can reduce temperature fluctuations to which the gametes and/or embryos may be subjected and is known to reduce evaporation of the embryonic culture medium 218.

Once the petri dish 200 has been filled with embryonic culture media 218 and the oil overlay 220 if desired, the practitioner can insert water 226 into the reservoir 224 of the humidifier 219, filling the humidifier 219. The practitioner can pour water 226, or another suitable fluid source of humidity 226, into the opening 416 of the culture stage 208 to fill the reservoir 224 of the humidifier 219.

The petri dish 200 preferably includes a transparent construction so that the practitioner is readily able to view the water level within the reservoir 224 to ensure that a height of the water 226 within the reservoir 224 does not exceed a height of the inner wall 412 and outer wall 410 of the cellular culture stage 208. Therefore, the gametes and/or embryos housed within the wells 216 inside the channel 414 are physically separated from the water 226 contained within the reservoir 224. As is illustrated in FIG. 2, the height of the water 226 within the reservoir 224 can approximate half the height or less of the inner wall 412 and outer wall 410, to reduce the likelihood that the gametes and/or embryos are subjected to contamination from the water 226 during movement of the petri dish 200.

The practitioner can then place gametes and/or embryos into the embryonic culture medium 218 located below the oil overlay 220. The gametes (e.g., egg and/or sperm cells) can be placed into the wells 216 in an unfertilized form, where fertilization will hopefully occur. Alternatively, a sperm injected egg can be placed into the well 216, as can be achieved through intracytoplasmic sperm injection (e.g., where a single sperm is manually injected into the egg prior to culture).

The practitioner can then place the lid 204 on the base 202, placing the petri dish 200 in a closed configuration 302. When in the closed configuration 302, the sidewall 402 of the lid 204 is located radially outwardly relative to the sidewall 404 of the base 202 and the upper rim 238 of the sidewall 404 contacts a lower surface 228 of the lid 204.

The petri dish 200 is now in a loaded configuration 234 (e.g., is now filled with a suitable liquid 226 and gametes and/or embryos have been placed into the embryonic culture medium 218 under the oil overlay 220) and is ready to be placed into an incubator (not shown) for the gametes and/or embryos to be cultured. As is standard practice in IVF clinics, the incubators can be set at 37° C., and the incubators can be filled with an appropriate gas mixture.

As the gametes and/or embryo are cultured, a portion of the water 226 contained within the reservoir 224 will evaporate into the atmosphere of the interior cavity 206, humidifying the interior cavity 206. It is believed that by maintaining and/or increasing the humidity of the atmosphere 240 within the interior cavity 206 may reduce the evaporation of the embryonic culture medium 218, thereby preserving the osmolality of the embryonic culture medium 218.

The integration of the humidifier 219 into the petri dish 200 advantageously enables the use of dry incubators (e.g., without a humidification system), which is believed to reduce the likelihood of culture contamination with bacteria and/or fungi from the incubator. Due to the transparent nature of the petri dish 200, the practitioner may routinely visibly inspect the water 226 level present within the reservoir 224. The practitioner may remove the lid 204 to add water 226 to the reservoir 224 if desired.

As has been described, the lid 204 includes a condensation director 232 which is designed to prevent condensation 508 from contaminating the culture in the wells 216 during the culture period. Should condensation 508 form on the lower surface 228 of the lid 204, the condensation 508 will hang from and flow downwardly along the inclined surface 214 over the channel 414. In this manner, the condensation director 232 is configured to substantially reduce and/or prevent condensation 508 from dripping from the lower surface 228 into the wells 216. Once the condensation 508 has flowed to the central surface 222 of the condensation director 232, the condensation 508 can drip off the central surface 222 of the condensation director 232 into the reservoir 224 below, essentially creating an artificial water cycle within the atmosphere 240.

Once the desired culture period has been reached, the practitioner can remove the petri dish 200 from the incubator. The lid 204 is removed from the base 202, providing access to the wells 216. The practitioner may then remove the embryos from the wells 216 to either be transferred into a patient or frozen for future use. The petri dish 200 can then either be disposed of (e.g., in the case of a single-use petri dish 200) or can be sanitized for future use.

In one exemplary, non-limiting form, a diameter 430 of the base 202 can approximate 60 mm, which is a commonly utilized size for petri dishes in IVF laboratories. However, it is contemplated that the base 202, and the petri dish 200, can be formed to various sizes and shapes, depending upon the type of cells to be cultured, and the desired parameters of the petri dish 200.

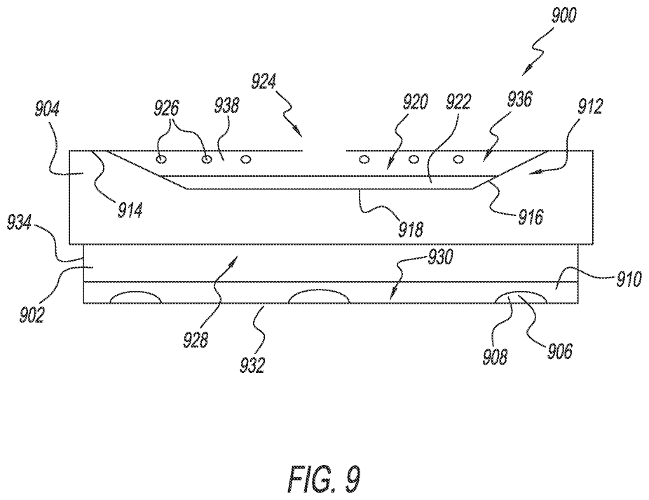

Referring now to FIG. 9, another form of an exemplary petri dish 900 will now be described. This petri dish 900 includes a base 902 and a lid 904. An interior cavity 928 is located between a lower surface 914 of the lid 904 and an interior surface 930 of the base 902.

A cell culture stage 932 is located at the interior surface 930 of the base 902. As is illustrated, at least one cell 908 to be cultured and a suitable culture medium 906 are placed on the cell culture stage 932. Depending upon the desired cells to be cultured, an oil overlay 910 can be placed in the base 902 to cover the culture medium 906 and cell 908 to be cultured.

The petri dish 900 includes a humidifier 912. The humidifier 912 is depicted as being located at the lid 904. The humidifier 912 can be integrally formed with the lid 904. As is illustrated, the humidifier 912 can hang from the lower surface 914 of the lid 904. However, it is also contemplated that the humidifier 912 can be a removable insert which includes an outer perimeter which will rest atop an upper rim (not shown) of a sidewall 934 of the base 902. In this form, the lid 904 can then be placed atop the removable insert.

The humidifier 912 includes a lower surface 918 and a sidewall 916. The lower surface 918 and the sidewall 916 of the humidifier 912 at least partially define a water reservoir 920. The water reservoir 920 is configured to house water 922.

A plurality of apertures 926 are located in an upper portion 936 of the sidewall 916 of the humidifier 912. These apertures 926 place the water reservoir 920 in flow communication with the interior cavity 928. The water reservoir 920 can be filled with water 922 to a height which is below the apertures 926, such that water 922 will not flow through the apertures 926 and into the interior cavity 928. The lid 904 can include an opening 924 through which water 922 can be poured to fill the water reservoir 920.

The humidifier 912 serves to humidify the interior cavity 928. Water 922 that is located within the water reservoir 920 will evaporate into the air 938. This humidified air 938 passes through the apertures 926 into the interior cavity 928, humidifying the atmosphere of the interior cavity 928. Condensation forming within the water reservoir 920 will remain in the water reservoir 920 and will drip downwardly into the water reservoir 920.

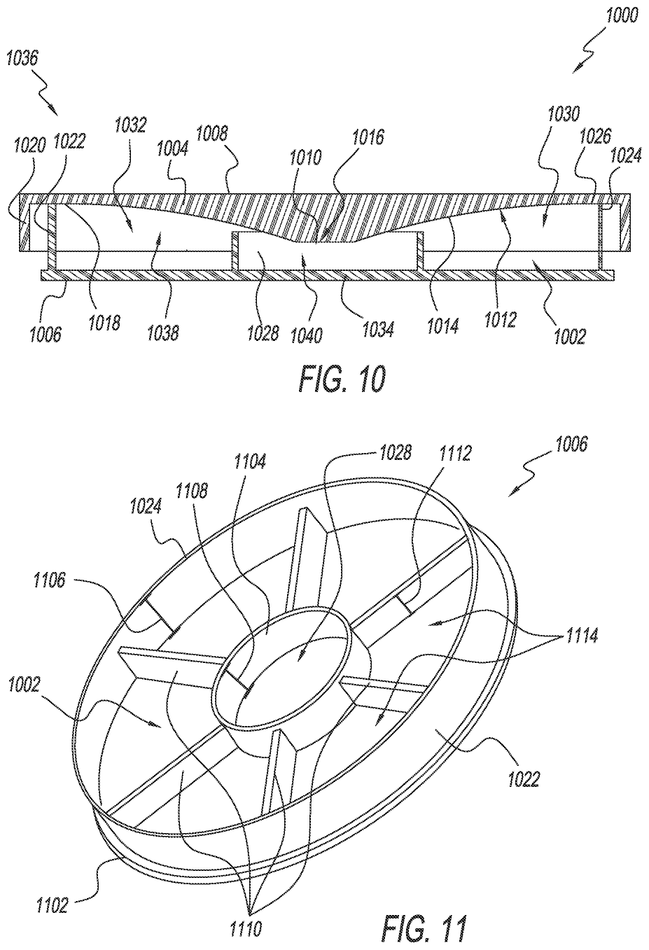

Referring now to FIG. 10, a further form of a humidified petri dish 1000 will now be described. The petri dish 1000 includes a base 1006 and a lid 1004. An interior cavity 1030 of the petri dish 1000 is enclosed when the lid 1005 is placed on the base 1006. The cells (not shown) to be cultured within the petri dish 1000 are placed on a cellular culture area, depicted as cell culture stage 1002.

The lid 1004 includes an upper surface 1008, a lower surface 1026, and a downwardly extending sidewall 1020. The base 1006 includes a lower surface 1034 and an outer wall 1022. The petri dish 1000 is depicted as having an overall cylindrical, puck-like shape 1036 when the lid 1004 is placed on the base 1006. When placed on the base 1006, the downwardly extending sidewall 1020 of the lid 1004 extends outwardly relative the outer wall 1022 and extends downwardly below the upper rim 1024 outer wall 1022.

As was described with sealing member 203 of the petri dish 200, a sealing member (not shown) can be disposed between the lid 1004 and the base 1006. Use of a sealing member can advantageously confine the atmosphere 1032, and the water vapor therein, within the sealed interior cavity 1030. One or more sealing members can be located on the upper rim 1024 of the outer wall 1022 and/or the lower surface 1026 of the lid 1004 to provide sealing engagement between the lid 1004 and the base 1006. Additionally and/or alternatively, a sealing member can be located between the outer wall 1022 of the base 1006 and the downwardly extending sidewall 1020 of the lid 1004.

Referring now to FIGS. 10-11, the base 1006 includes a reservoir 1028. The reservoir 1028 can be centrally located in the base 1006 (e.g., located toward a center and/or midpoint of the base 1006). A reservoir wall 1104 extends upwardly from the lower surface 1034 and defines an outer perimeter of the reservoir 1028. The outer wall 1022 and the reservoir wall 1104 can include a substantially circular forms when viewed from above. The reservoir wall 1104 can extend concentrically relative the outer wall 1022.

The reservoir 1028 can be partially filled with water (not shown) prior to loading the petri dish 1000. The reservoir 1028 is configured to serve as an evaporative humidifier 1040 (e.g., with water from the reservoir 1028 evaporating into the atmosphere 1032 that is preferably sealed within the interior cavity 1030). The humidifier 1040 is configured to maintain and/or increase a humidity of the atmosphere 1032 within the interior cavity 1030.

As was previously discussed with regard to the humidifier 219 and the petri dish 200, maintaining a desired level of humidity within the atmosphere 1032 above the cell culture stage 1002 can be especially advantageous for applications involving IVF. Specifically, maintaining a desired level of humidity within the atmosphere 1032 will reduce evaporation of culture medium during the desired culture period, and will help maintain the osmolality of the culture medium.

The reservoir 1028 and the cell culture stage 1002 can be integrally formed with the base 1006. The cell culture stage 1002 is depicted as extending between the reservoir wall 1104 and the outer wall 1022. The reservoir wall 1104 serves as a physical separation between the cell culture stage 1002 and any water within the reservoir 1028. The cell culture stage 1002 can include a plurality of wells 1114. Dividers 1110 can be disposed in the cell culture stage 1002 to serve as separations between the plurality of wells 1114. The dividers 1110 are depicted as extending between the reservoir wall 1104 and the outer wall 1022, along the lower surface 1034. The cell culture stage 1002 is depicted as including six wells 1114; however, it is contemplated that the cell culture stage 1002 can include any desired number of wells 1114 by increasing or decreasing the number of dividers 1110 disposed therein.

In one exemplary form, a height 1112 of the dividers 1110 can be less than a height 1108 of the reservoir wall 1104 and less than a height 1106 of the outer wall 1022. The lower height 1112 of the dividers 1110 can enable an oil overlay (not shown) to continuously extend over cell culture stage 1002 above the dividers 1110, with the oil overlay confined in the cell culture stage 1002 between the reservoir wall 1104 and the outer wall 1022. This continuous oil overlay can permit a practitioner to easily transfer gametes and/or embryos between the wells 1114, without the gametes and/or embryos directly contacting the external atmosphere.

A condensation director 1012 is located at the lid 1004. The condensation director 1012 is depicted as being integrally formed into a lower surface 1026 of the lid 1004. The condensation director 1012 extends over the cell culture stage 1002. The condensation director 1012 is configured direct condensation forming on the lower surface 1026 into the reservoir 1028. The condensation director 1012 is configured to prevent condensation from dripping into the cell culture stage 1002 and contaminating the culture therein.

The condensation director 1012 includes an incline 1014. The incline 1014 of the condensation director 1012 extends downwardly from an outer portion 1018 of the lid 1004 toward a central portion 1016 of the lid 1004. The incline 1014 of the condensation director 1012 is depicted as taking the form of a downward curvature 1038.

The incline 1014 of the condensation director 1012 over the cell culture stage 1002 should be of a sufficient magnitude such that condensation will flow downwardly along the condensation director 1012 toward the central portion 1016 of the lid 1004 without dripping therefrom. Specifically, the incline 1014 should include a sufficiently steep curvature, angle, and/or average angle, such that condensation droplets will begin to flow down along the condensation director 1012, prior to the droplets increasing in size and dripping into the cell culture stage 1002 below.

Condensation droplets which form on the lower surface 1026 of the lid 1004 will flow downwardly along the condensation director 1012, with the condensation droplets remaining adhered to the condensation director 1012. The condensation director 1012 serves as a flowpath for condensation droplets to remain engaged with, and flow downwardly along, as the droplets pass over the cell culture stage 1002. In this manner, the condensation director 1012 enables the condensation droplets to cross above the cell culture stage 1002 while hanging from and remaining adhered to the condensation director 1012. Once the condensation droplets have passed over the cell culture stage 1002, the condensation droplets will reach the lower surface 1010 of the condensation director 1012 and will drip therefrom, into the reservoir 1028 below.

The base 1006 is depicted as including a lower extension 1102. This lower extension 1102 can provide stability to the base 1006. The lower extension 1102 can serve as a grasping tab which a user may grasp to move the petri dish 1000. It is also contemplated that a second extension (not shown) can be located above the lower extension 1102 extending around the outer wall 1022 in a manner similar to the lower extension 1102. In this manner, a user can securely grip the outer wall 1022 with their fingers located between the lower extension 1102 and the second extension.

The base 1006, including the reservoir wall 1104 and dividers 1110, can be injection molded of medical grade polystyrene. The lid 1004 and condensation director 1012 can be injection molded of medical grade polystyrene. However, it is contemplated that the base 1006, the cell culture stage 1002, the lid 1004, the condensation director 1012, and other features discussed herein may be formed utilizing a variety of techniques, from a variety of polymers or glasses, as are known within the art of petri dish construction. The petri dish 1000 can be prepackaged in sterile medical packaging

An outer diameter of petri dishes 200, 900, and/or 1000 can approximate 60 mm. A total height of the petri dishes 200, 900, and/or 1000 can be less than 20 mm, and can approximate 15 mm. These exemplary, non-limiting dimensions are similar to petri dishes currently utilized in IVF laboratories; therefore, the use of such dimensions can enable the petri dishes 200, 900, and/or 1000 to fit into smaller incubators.

Petri dishes 200, 900, and 1000 have been discussed herein with regard to the culture of gametes and/or embryos, and are believed to be well suited for use during IVF. However, it is contemplated that the petri dishes 200, 900, and 1000 can be utilized for the culture of a variety of cells.

Additionally, the petri dishes 200, 900, and 1000 have been described as including specific forms and features. However, it is contemplated that petri dishes embodying the teachings of the present application may include any one or more of these forms and features, and it is contemplated that various forms and features can be interchanged between the petri dishes 200, 900, and 100, depending upon the desired design parameters of the petri dish. For example, it is contemplated that the lid 204 (including the flow director 232) of the petri dish 200 can be utilized in connection with the base 1006 (including the integrally formed cell culture stage 1002 and reservoir 1028) of the petri dish 1000. In this non-limiting example, the lid 204 will removably couple with the base 1006.

While the invention has been described in connection with what is presently considered to be the most practical and preferred embodiment, it is to be understood that the invention is not to be limited to the disclosed embodiment(s), but on the contrary, is intended to cover various modifications and equivalent arrangements included within the spirit and scope of the appended claims, which scope is to be accorded the broadest interpretation so as to encompass all such modifications and equivalent structures as permitted under the law.

It should be understood that while the use of the word preferable, preferably, or preferred in the description above indicates that feature so described may be more desirable, it nonetheless may not be necessary and any embodiment lacking the same may be contemplated as within the scope of the invention, that scope being defined by the claims that follow.

In reading the claims it is intended that when words such as “a,” “an,” “at least one” and “at least a portion” are used, there is no intention to limit the claim to only one item unless specifically stated to the contrary in the claim. Further, when the language “at least a portion” and/or “a portion” is used the item may include a portion and/or the entire item unless specifically stated to the contrary.

What is claimed is:

1. An apparatus, comprising:
a cellular culture container having an interior cavity;
a cellular culture area located in the interior cavity, wherein the cellular culture area is configured to receive at least one cell to be cultured; and
a condensation director.

2. The apparatus of claim 1, further comprising a reservoir that is physically separated from the cellular culture area, and wherein the condensation director is configured to direct condensation toward the reservoir.

3. The apparatus of claim 1, wherein the cellular culture container includes a lid configured to removably cooperate with a base, and wherein the condensation director is located at the lid.

4. The apparatus of claim 3, wherein the condensation director is integrally formed with the lid, and wherein a lower surface of the lid at least partially defines the condensation director.

5. The apparatus of claim 3, further comprising a reservoir located in the base, wherein the condensation director at least partially extends over the cellular culture area, wherein the condensation director includes an inclined surface, and wherein the condensation director is configured to provide for the flow of condensation along the inclined surface toward the reservoir.

6. The apparatus of claim 5, wherein the inclined surface is disposed at an angle greater than 3 degrees.

7. The apparatus of claim 5, wherein the inclined surface includes a downwardly directed curvature.

8. The apparatus of claim 5, further comprising a sealing member located between the base and the lid, wherein the reservoir further comprises an evaporative humidifier, and wherein the sealing member is configured to prevent the egress of water vapor from the interior cavity.

9. A petri dish, comprising:
an outer housing having an interior cavity;
a cell culture stage located in the interior cavity, wherein the cell culture stage is configured to receive a culture; and
a condensation director configured to direct condensation toward a reservoir.

10. The petri dish of claim 9, wherein the outer housing includes a base and a lid, wherein the condensation director is located at the lid, and wherein the reservoir is located at the base.

11. The petri dish of claim 10, wherein the reservoir is configured to humidify a substantially sealed atmosphere confined in the interior cavity.

12. The petri dish of claim 11, wherein the condensation director is integrally formed with the lid, wherein the condensation director at least partially extends over the cell culture stage, wherein the condensation director includes an inclined surface, and wherein the condensation director is configured to prevent condensation from dripping into the cell culture stage.

13. The petri dish of claim 12, wherein the inclined surface includes a curvature.

14. The petri dish of claim 9, wherein the outer housing includes a base, wherein the cell culture stage and reservoir are integrally formed into the base, and wherein the reservoir is located at a central portion of the base.

15. The petri dish of claim 14, further comprising a reservoir wall which defines an outer perimeter of the reservoir, wherein the cell culture stage includes a plurality of culture wells, and wherein each culture well is configured to receive a suitable culture medium and at least one of a gamete, a fertilized gamete, and/or an embryo.

16. The petri dish of claim 15, wherein the culture wells are separated by a plurality of dividers extending between the reservoir wall and an outer wall of the base, and wherein a height dimension of the dividers is less than a height dimension of the reservoir wall.

17. A petri dish, comprising:
a lid configured to removably couple with a base;
an internal cavity at least partially defined between the lid and the base, wherein the lid and the base sealingly cooperate to reduce and/or prevent the egress of water vapor from the internal cavity to an external environment;
a cell culture stage located in the internal cavity, wherein the cell culture stage is configured to receive at least one of a gamete, a fertilized gamete, and/or an embryo; and
a humidifier in fluid communication with the internal cavity.

18. The petri dish of claim 17, wherein the humidifier is a reservoir, and wherein the reservoir is located in a central portion of the base.

19. The petri dish of claim 18, wherein a condensation director is located at a lower surface of the lid, wherein the condensation director includes an inclined surface which extends downwardly toward the central portion of the base, and wherein the condensation director is configured to direct condensation into the reservoir.

20. The petri dish of claim 19, wherein the condensation director at least partially extends over the cell culture stage, and wherein the condensation director provides for condensation to flow downwardly along the inclined surface, absent condensation dripping from the inclined surface onto the cell culture stage.

21. The petri dish of claim 19, wherein the cell culture stage is integrally formed in the base, and wherein the condensation director is integrally formed in the lid.

22. The petri dish of claim 17, further comprising a sealing member located between the base and the lid.
